(19) United States
(12) Patent Application Publication
(10) Pub. No.: US 2026/0317722 A1
(43) Pub. Date: Oct. 15, 2026

(54) METHODS OF DIFFERENTIATION OF PLURIPOTENT STEM CELLS INTO MESENCHYMAL STROMAL CELLS

(71) Applicant: R.P. SCHERER TECHNOLOGIES, LLC, Carson City, NV (US)

(72) Inventors: Smita SUDHEER, Carson City, NV (US); Boris GREBER, Carson City, NV (US)

(21) Appl. No.: 19/487,416

(22) PCT Filed: Feb. 29, 2024
(86) PCT No.: PCT/US2024/017873

§ 371 Date: Nov. 24, 2025

Related U.S. Application Data
(60) Provisional application No. 63/449,513, filed on Mar. 2, 2023.

(51) Int. Cl. C12N 5/0775

(52) U.S. Cl. CPC C12N 5/0668; C12N 2500/99; C12N 2501/415; C12N 2501/727; C12N 2506/45; C12N 2533/52

(57) ABSTRACT

The invention provides a method of producing a population of CD73⁺ CD44⁺, CD90⁺ mesenchymal stromal cells (MSCs). The CD73⁺ CD44⁺, CD90⁺ MSCs are used in methods of generating terminally differentiated osteogenic, adipogenic and chondrogenic cells from pluripotent stem cells (PSCs). The differentiation method includes the use of a single agent, WNT signaling pathway activator such as a GSK3β inhibitor, used on adherent culture of PSCs.

### CROSS-REFERENCE TO RELATED APPLICATIONS

The present application claims the benefit of priority under U.S.C. 119(e) to U.S. Provisional Application No. 63/449,513, filed Mar. 2, 2023, the entire contents of which is incorporated herein by reference in its entirety.

### BACKGROUND OF THE INVENTION

### Field of the Invention

The present invention relates generally to mesenchymal stromal cells (MSCs), and more specifically to methods of generating MSCs from pluripotent stem cells (PSCs).

### Background Information

Pluripotent stem cells are cells that are capable of self-renewing and to giving rise to all cells of the three primary groups of cells that make up a human body, including: ectoderm (skin and nervous system cells), endoderm (including gastrointestinal and respiratory tracts cells, endocrine glands cells, liver cells, and pancreas cells), and mesoderm (including bone, cartilage, most of the circulatory system cells, muscles cells, connective tissue cells, and more). Pluripotent stem cells can be induced pluripotent stem cells (iPSCs) or embryonic stems cells (ESCs). Because they can propagate indefinitely and give rise to every cell type in the body, they represent a potential source for the development of therapeutic cells. Among many other cell types, PSCs can be differentiated into mesenchymal stromal cells, with the potential to generate osteogenic, adipogenic and chondrogenic cells.

Mesenchymal stromal cells (MSCs) are self-renewing adult multipotent stem cells that are present in various tissues such as bone marrow, adipose tissue, placenta, dental and the umbilical cord, probably in all vascularized human tissues, in low numbers. MSCs have been found to have therapeutic effects on several diseases. The International Society for Cellular Therapy (ISCT) has issued minimal criteria to define multipotent MSCs: plastic adherence, expression of cell surface markers, CD73, CD90, CD105, lack of expression of other lineage markers, in vitro tri-lineage differentiation potential.

Currently the MSCs derived from the bone marrow are used for treating diseases. At present there are more than 1400 clinical trials registered on the clinicaltrials.gov website using MSCs for the treatment of various human diseases and medical conditions. However, the limited supply of bone marrow-derived MSCs is a hindrance to their successful use in cell-replacement therapy. This shortcoming can be met by Pluripotent Stem Cells (PSCs) that have considerable advantages over MSCs, owing to their unlimited proliferation and multi-lineage differentiation capacity. The use of human PSCs (hPSCs) in either regenerative medicine or disease modelling/drug discovery requires their directed differentiation to a pure population of the cell type, avoiding other cell types. Differentiation of PSCs towards MSCs can be a valuable method to generating ample cells for applications such as regenerative therapy for musculoskeletal disorders, disease modelling or drug screening.

Various methods have been adopted to derive MSCs from hPSCs. The protocols usually differentiate the hiPSCs to a mesoderm-like state and further differentiate them to MSCs. For example, Human PSCs have been differentiated to MSCs (i) with a temporal induction of neural ectoderm using CHIR-99021 and SB-431542 in chemically defined media, followed by the exposure to the conventional MSC growth medium, containing bovine serum; (ii) by directly exposing the cells to the conventional medium that is used for growing MSCs: the medium containing 10% bovine serum; or (iii) using the TGF/ACTIVIN/NODAL inhibitor, SB431542 for the initial differentiation of hPSCs to MSCs.

Those protocols used to differentiate hPSCs to MSCs use either non-adherent (embryoid bodies) or adherent methods. Most make embryoid bodies from hiPSCs, before differentiating them into MSCs and utilize a complex procedure, that includes several small molecules and/or recombinant proteins.

There remains a need in the art for a simpler and efficient protocol for MSC induction.

### SUMMARY OF THE INVENTION

The present invention is based on the seminal discovery that the sole use of a WNT signaling pathway activator, such as a GSK3β inhibitor on PSCs yields enriched cultures of MSCs that can further yield terminally differentiated osteogenic, adipogenic and chondrogenic cells.

In one embodiment, the present invention provides a method of generating mesenchymal stromal/stem cells (MSCs) including: (a) contacting an adherent culture of pluripotent stem cells (PSCs) with a WNT signaling pathway activator for about 4-8 days; and (b) expanding the cells in culture from (a) in the absence of the WNT signaling pathway inhibitor, thereafter, thereby generating MSCs.

In one aspect, the WNT signaling pathway activator is a GSK3β inhibitor. In some aspects, the GSK3β inhibitor is CHIR99021. In one aspect, the PSCs are contacted with about 4 μM of CHIR99021. In another aspect, the PSCs are contacted with CHIR99021 for about 6 days. In various aspects, the PSCs are contacted with about 4 μM of CHIR99021 for about 6 days. In one aspect, contacting comprises incubating the PSCs with fewer than three WNT signaling pathway activators. In another aspect, contacting comprises incubating the PSCs with a single WNT signaling pathway activator. In one aspect, prior to (a), the PSCs are maintained in a culture media including a ROCK inhibitor. In some aspects, the ROCK inhibitor is Y-27632. In another aspect, the PSCs are cultured on a laminin-coated surface. In one aspect, expanding the cells includes culturing the cells on a non-coated surface. In another aspect, the method produces an enriched culture of MSCs. In some aspects, the enriched culture includes at least about 90% MSCs. In other aspects, the enriched culture includes at least about 95% MSCs. In one aspect, the MSCs are CD73, CD44 and CD90 positive. In another aspect, the MSCs are CD14, CD31, TRA 1-60, CD34, HLA-DR, SOX2, miR-302 HT, PECAM1, CDH5, CD45, MYH6 and SOX10 negative. In one aspect, the MSCs generated are multipotent stem cells with osteogenic, adipogenic and chondrogenic differentiation potential. In another aspect, expanding the cells includes culturing the cells in a MSCs supporting culture media. In one aspect, contacting and expanding the cells include contacting and expanding in serum-free culture conditions. In another aspect, the culture of PSCs does not include embryoid bodies. In one aspect, the WNT signaling pathway activator is a WNT ligand, a recombinant protein, an inhibitor of a WNT signaling pathway inhibitor or an indirect WNT activator. In one aspect, the PSCs are human pluripotent stem cells (hPSCs). In some aspects, the hPSCs are induced pluripotent stem cells (hiPSCs) or human embryonic stems cells (hESCs). In one aspect, the PSCs are iPSCs.

In another embodiment, the invention provides a method of generating mesenchymal stromal/stem cells (MSCs) including: (a) contacting an adherent culture of iPSCs with about 4 μM of a single GSK3β inhibitor for about 6 days; and (b) expanding the cells in culture from (a) in the absence of the GSK3β inhibitor thereafter, wherein after (b), a population of at least about 90% pure CD73+, CD44+ and CD90+MSCs is obtained, thereby obtaining MSCs.

### BRIEF DESCRIPTION OF THE DRAWINGS

FIG. 1 is a schematic representation of a method of the invention.

FIG. 2 shows photographs illustrating MSCs obtained by a method of the invention in continuous culture (left) and after being frozen thawed (right).

FIG. 3 is a graph illustrating MSCs growth kinetics in culture.

FIGS. 4A-4C illustrate characterization of the MSCs. FIG. 4A shows histograms illustrating CD73, CD90, CD44 and CD105 expression on MSCs as evaluated by flow cytometry. FIG. 4B shows histograms illustrating CD14, CD45, CD31, TRA 1-60, CD34 and HLA-DR expression in MSCs as evaluated by flow cytometry. FIG. 4C shows histograms illustrating negative controls staining on MSCs as evaluated by flow cytometry.

FIGS. 5A-5E illustrate the expression of various alternative markers on iPSCs, MSCs, EPC, HSCs, and CMs, as measured by RT-qPCR. FIG. 5A is a graph bar illustrating the expression of pluripotency genes. FIG. 5B is a graph bar illustrating the expression of endothelial markers. FIG. 5C is a graph bar illustrating the expression of hematopoietic marker. FIG. 5D is a graph bar illustrating the expression of cardiac marker. FIG. 5E is a graph bar illustrating the expression of neural crest marker.

FIG. 6 shows illustrative photographs of adipogenic, osteogenic and chondrogenic cells differentiated from MSCs.

FIGS. 7A-7B illustrate the differentiation of MSCs in various culture media. FIG. 7A shows histograms illustrating CD73, CD44 and CD90 expression on MSCs differentiated in MSC expansion medium 1. FIG. 7B shows histograms illustrating CD73, CD44 and CD90 expression on MSCs differentiated in MSC expansion medium 2.

FIG. 8 shows photographs illustrating MSCs derived from 3 independent iPSCs lines.

FIG. 9 shows histograms illustrating CD73, CD90, CD44 and CD105 expression on 3 independent MSC lines as evaluated by flow cytometry.

FIG. 10 shows histograms illustrating TRA 1-60, CD31, CD45, CD14, CD34 and HLA-DR expression on MSCs as evaluated by flow cytometry.

### DETAILED DESCRIPTION OF THE INVENTION

The present invention is based on the seminal discovery that that the sole use of a WNT signaling pathway activator, such as a GSK3β inhibitor on PSCs yields enriched cultures of MSCs that can further yield terminally differentiated osteogenic, adipogenic and chondrogenic cells.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a method of generating mesenchymal stromal/stem cells (MSCs) including: (a) contacting an adherent culture of pluripotent stem cells (PSCs) with a WNT signaling pathway activator for about 4-8 days; and (b) expanding the cells in culture from (a) in the absence of the a WNT signaling pathway activator, thereafter, thereby generating MSCs.

The methods described herein provide cellular culture conditions in which human pluripotent stem cells are grown, that yield the generation of a population of mesenchymal stromal/stem cells (MSCs).

Stem cells are undifferentiated cells that have the ability to self-renew indefinitely and to remain in said undifferentiated state. As opposed to embryonic stem cells which can only be isolated from the inner mass of a blastocyst, there are three known accessible sources of adult stem cells: the bone marrow which requires the drilling of a bone, the adipose tissue which is accessible by liposuction, and the blood, from which the cells can be extracted among other cells. The term “pluripotent stem cells”, as used herein refers to cells that are capable of generating all the cell types of an organism, i.e., cells derived from any of the three germ layers. On the other hand, multipotent stem cells can differentiate into several cell type, but only those of a closely related family of cells, generally the cell types of the organ from which they originate. Most adult stem cells are multipotent but small amounts of pluripotent adult stem cells can be retrieved from umbilical cord or other tissues. The sources of cells used for cell therapy include stem cells such as embryonic stem cells (ESCs), adult stem cells, and induced pluripotent stem cells (iPSCs).

In some aspects, the PSCs used in the methods described herein are human (hPSCs), and in some instances the human PSCs are induced pluripotent stem cells (hiPSCs) or human embryonic stems cells (hESCs). In various aspects, the PSCs are human induced pluripotent stem cells (hiPSCs).

By “generating” or “producing” MSCs, it is meant that the present methods provide physical and chemical culture conditions that have been optimized to induce the differentiation of iPSCs into MSCs. The differentiation method described herein yields a cell population that is enriched for MSCs. For example, greater than 80%, greater than 85%, greater than 90%, greater than 95%, 96%, 97%, 98% or 99% MSCs are obtained in short times and using convenient cultures conditions.

Physical culture conditions include but are not limited to the culture environment of the cell (e.g., adherent versus suspension culture, or in two-dimensional versus in three-dimensional culture systems), the pH of the culture media, the gas concentration in the incubator (e.g., CO2 concentration, O2 concentration), and the temperature.

There are two basic systems for growing cells in culture, as monolayers on an artificial substrate (i.e., adherent culture) or free-floating in the culture medium (suspension culture). Most of the cells derived from vertebrates, except for hematopoietic cell lines and a few others, are anchorage-dependent and have to be cultured on a suitable substrate that is specifically treated to allow cell adhesion and spreading (i.e., tissue-culture treated). However, many cell lines can also be adapted for suspension culture.

In various aspects, the culture of PSCs is an adherent layer of cells. In some aspects, the layer of cells is grown in a two-dimensional culture system or on microcarriers.

PSCs can be cultured in a three-dimensional culture system, by relying on non-adherent conditions and the formation of embryoid bodies. In one aspect, in the methods described herein, the culture of PSCs does not include embryoid bodies.

Physical culture conditions include the gas concentration in the incubator. Incubation of cell cultures is typically performed in normal atmosphere with 15-22% oxygen and 5% CO₂ for expansion and seeding. In various aspects, the PSCs are grown in a humidified atmosphere including about 5% CO₂ concentration, and normoxic conditions (non-hypoxic O₂ concentration). While hypoxic culture conditions are thought to support stem cell performance in general, in the present methods, the PSCs are cultured under conditions that are not hypoxic conditions. As used herein, “normoxic” conditions refer to culture conditions including atmospheric O₂ concentration (e.g., about 15-25% O₂ concentration). As used herein, hypoxic conditions are characterized by a lower oxygen concentration as compared to the oxygen concentration of ambient air (approximately 15%-25% oxygen).

Chemical culture conditions include but are not limited to the agents or molecules that are added to the culture medium to achieve the desired effects sought after (i.e., differentiation of PSCs into MSCs). The terms “agent” and “molecule” are used interchangeably and include, but are not limited to, small molecules (including small molecules that do not have optimal cell-permeability), lipids, nucleosides, nucleotides, nucleic acids, polynucleotides, oligonucleotides, antibodies, toxins, negatively charged polymers and other polymers, for example proteins, peptides, hormones, carbohydrates, or polyamines.

In the methods described herein, the PSCs are contacted with an agent that is a “a WNT signaling pathway activator”. By “contacting” it is meant that the cells are cultured with the one or more agent of interest. That is the cells are cultured in their regular culture media, in which a desired concentration of one or more agent of interest is added. For examples, the cells are cultured with a WNT signaling pathway activator.

A “pathway signaling activator” as used herein refers to any molecule that can activate, enhancing, or inducing a signaling pathway of interest. A signaling pathway is a series of chemical reactions in which a group of molecules in a cell work together to control a cell function, such as cell differentiation. A cell receives signals from its environment when a molecule, such as a hormone or growth factor, binds to a specific protein receptor on or in the cell. After the first molecule in the pathway receives a signal, it activates another molecule. This process is repeated through the entire signaling pathway until the last molecule is activated and the cell function is carried out. Abnormal activation of signaling pathways, or inhibition of a signaling pathway may lead to diseases, or, in the case of pluripotent cells to alteration of the pluripotent state, and therefore to differentiation. The term “molecule” includes, but is not limited to, small molecules (including small molecules that do not have optimal cell-permeability), lipids, nucleosides, nucleotides, nucleic acids, polynucleotides, oligonucleotides, antibodies, toxins, negatively charged polymers and other polymers, for example proteins, peptides, hormones, carbohydrates, or polyamines. Non-limiting examples of polynucleotides include short interfering nucleic acid (siNA), antisense, enzymatic nucleic acid molecules, 2′,5′-oligoadenylate, triplex forming oligonucleotides, aptamers, and decoys. Biologically active molecules include antibodies (e.g., monoclonal, chimeric, humanized etc.), cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, allozymes, aptamers, decoys and analogs thereof, and small nucleic acid molecules, such as short interfering nucleic acid (siNA), short interfering RNA (siRNA), double-stranded RNA (dsRNA), micro-RNA (miRNA), antagomirs, and short hairpin RNA (shRNA) molecules.

The WNT signaling pathways are a group of signal transduction pathways which begin with proteins that pass signals into a cell through cell surface receptors. Wnt signaling pathways use either nearby cell-cell communication (paracrine) or same-cell communication (autocrine). Three Wnt signaling pathways have been characterized: the canonical Wnt pathway, the noncanonical planar cell polarity pathway, and the noncanonical Wnt/calcium pathway. All three pathways are activated by the binding of a Wnt-protein ligand to a Frizzled family receptor, which passes the biological signal to the Dishevelled protein inside the cell. The canonical Wnt pathway leads to regulation of gene transcription and is thought to be negatively regulated in part by the SPATS1 gene. The noncanonical planar cell polarity pathway regulates the cytoskeleton that is responsible for the shape of the cell. The noncanonical Wnt/calcium pathway regulates calcium inside the cell. Wnt signaling was first identified for its role in carcinogenesis, then for its function in embryonic development. The embryonic processes it controls include body axis patterning, cell fate specification, cell proliferation and cell migration. These processes are necessary for proper formation of important tissues including bone, heart, and muscle. Its role in embryonic development was discovered when genetic mutations in Wnt pathway proteins produced abnormal fruit fly embryos. Later research found that the genes responsible for these abnormalities also influenced breast cancer development in mice. Wnt signaling also controls tissue regeneration in adult bone marrow, skin, and intestine.

In one aspect, the WNT signaling pathway activator is a WNT ligand, a recombinant protein, an inhibitor of a WNT signaling pathway inhibitor or an indirect WNT activator.

Many substances can activate WNT signaling pathway (e.g., activate beta-catenin): non-limiting examples of WNT signaling pathway activator include WNT ligands, GSK3 inhibitors, Axin inhibitors, APC inhibitors.

### WNT Activators Also Include:

1. Recombinant Proteins. There are several WNT ligands that can activate WNT signaling. WNT3a is the most commonly used WNT ligand. Recombinant Wnt1, WNT2, Wnt3a, and Wnt7a are other available options. Another protein, Norrin is not related to Wnt family proteins, but it induces activation of the canonical Wnt signaling pathway.
2. Inhibition of WNT inhibitors. A commonly used option is R-spondin.
3. Indirect WNT activators: GSK-30 inhibitors:
a. LiCl is commonly used.
b. Indirubins and derivatives: 6-bromo-indirubin-30-oxime (6-BIG) is commonly used.
c. Small molecules: CHIR99021 is commonly used. Some others include SB-216763 and SB-415286.

Acceptable concentrations range for the application described herein is dependent upon the WNT signaling pathway activator, and one of skill in the art would easily determine such concentration range. In general, for WNT ligands, an appropriate concentration range is about 1-500 ng/ml, preferably up to 200 ng/ml. For example, WNT3a is usually used in a range of about 1-200 ng/ml, preferably above 10 ng/ml; LiCl is usually used in a range of about 1-10 mM; CHIR99021 is usually used in a range of about 0.1-10 μM; R-spondin is usually used in a range of about 1-200 ng/ml; 6-bromoindirubin-3′-oxime (BIO) is usually used in a range of about 0.1-10 μM; Norrin is usually used in an range of about 1-200 ng/ml; SB415286 is usually used in a range of about 1-100 μM.

In some aspects, the WNT signaling pathway activator a GSK3 inhibitor.

In one aspect, the PSCs are contacted with about 2-6 μM of a GSK3β inhibitor. For example, the cells are contacted with about 1, 2, 3, 4, 5, 6, 7 or 8 μM of a GSK3β inhibitor. In various aspects, the PSCs are contacted with about 4 μM of a GSK3β inhibitor.

In another aspect, the PSCs are contacted with the GSK3β inhibitor for about 4-8 days. For example, the cells are contacted with the GSK3β inhibitor for about 2, 3, 4, 5, 6, 7, 8, 9 or 10 days. In various aspects, the PSCs are contacted with the GSK3β inhibitor for about 6 days.

In many aspects, the PSCs are contacted with about 4 μM of a GSK3β inhibitor for about 6 days.

There are many GSK3β inhibitors available. The methods described herein includes the use of a combination of GSK3β inhibitors. Non-limiting examples of GSK3β inhibitors include: 3F8, A 1070722, Alsterpaullone, AR-A 014418, AZD 2858, BIO, BIO-acetoxime, CHIR 98014, CHIR 99021, CHIR 99021 trihydrochloride, Indirubin-3′-oxime, Kenpaullone, Lithium carbonate, Lithium Chloride, SB 216763, SB 415286, TC-G 24, TCS 2002, TCS 21311, TDZD 8 and TWS 119.

In one aspect, contacting comprises incubating the PSCs with fewer than three GSK3β inhibitors. For example, the method includes contacting PSCs with a combination of two different GSK3β inhibitors. In another aspect, contacting comprises incubating the PSCs with a single GSK3β inhibitor. In various aspects, the GSK3β inhibitor is CHIR99021.

In one aspect, the PSCs are contacted with about 2-6 μM of CHIR99021. For example, the cells are contacted with about 1, 2, 3, 4, 5, 6, 7 or 8 μM of CHIR99021. In various aspects, the iPSCs are contacted with about 4 μM of CHIR99021.

In another aspect, the PSCs are contacted with CHIR99021 for about 4-8 days. For example, the cells are contacted with CHIR99021 for about 2, 3, 4, 5, 6, 7, 8, 9 or 10 days. In various aspects, the PSCs are contacted with CHIR99021 for about 6 days.

In many aspects, the PSCs are contacted with about 4 μM of CHIR99021 for about 6 days.

In one aspect, prior to (a), the PSCs are maintained in a culture media including a ROCK inhibitor. In some aspects, the ROCK inhibitor is Y-27632.

In the methods described herein, the PSCs are cultured as an adherent layer of cells. The adherent culture is grown in a two-dimensional culture system or on microcarriers.

For example, the PSCs are cultured on scaffold composed of microcarriers, which are beads or particles. The beads may be microscopic or macroscopic and may further be dimensioned to permit penetration into tissues or compacted to form a particular geometry. In some aspects, the framework for the cell cultures comprises particles that, in combination with the cells, form a three-dimensional tissue. The cells attach to the particles and to each other to form a three-dimensional tissue. Beads or microcarriers are typically considered a two-dimensional system or scaffold. As used herein, a “microcarriers” refers to a particle having size of nanometers to micrometers, where the particles may be any shape or geometry, being irregular, non-spherical, spherical, or ellipsoid. The size of the microcarriers suitable for the purposes herein can be of any size suitable for the particular application. In some embodiments, the size of microcarriers suitable for the three-dimensional tissues may be those administrable by injection. In some embodiments, the microcarriers have a particle size range of at least about 1 μm, at least about 10 μm, at least about 25 μm, at least about 50 μm, at least about 100 m, at least about 200 μm, at least about 300 μm, at least about 400 μm, at least about 500 μm, at least about 600 μm, at least about 700 μm, at least about 800 μm, at least about 900 μm, at least about 1000 μm.

In some aspects in which the microcarriers are made of biodegradable materials. In some aspects, microcarriers comprising two or more layers of different biodegradable polymers may be used. In some embodiments, at least an outer first layer has biodegradable properties for forming the three-dimensional tissues in culture, while at least a biodegradable inner second layer, with properties different from the first layer, is made to erode when administered into a tissue or organ.

In some aspects, the microcarriers are porous microcarriers. Porous microcarriers refer to microcarriers having interstices through which molecules may diffuse in or out from the microparticle. In other embodiments, the microcarriers are non-porous microcarriers. A nonporous microparticle refers to a microparticle in which molecules of a select size do not diffuse in or out of the microparticle.

Microcarriers for use in the compositions are biocompatible and have low or no toxicity to cells. The microcarriers may comprise various polymers, natural or synthetic, charged (i.e., anionic, or cationic) or uncharged, biodegradable, or nonbiodegradable. The polymers may be homopolymers, random copolymers, block copolymers, graft copolymers, and branched polymers.

In some aspects, the microcarriers comprise non-biodegradable microcarriers. Non-biodegradable microcapsules and microcarriers include, but not limited to, those made of polysulfones, poly (acrylonitrile-co-vinyl chloride), ethylene-vinyl acetate, hydroxyethyl methacrylate-methyl-methacrylate copolymers. These are useful to provide tissue bulking properties or in embodiments where the microcarriers are eliminated by the body.

In some aspects, the microcarriers comprise degradable scaffolds. These include microcarriers made from naturally occurring polymers, non-limiting example of which include, among others, fibrin, casein, serum albumin, collagen, gelatin, lecithin, chitosan, alginate, or poly-amino acids such as poly-lysine. In other aspects, the degradable microcarriers are made of synthetic polymers, non-limiting examples of which include, among others, polylactide (PLA), polyglycolide (PGA), poly(lactide-co-glycolide) (PLGA), poly(caprolactone), polydioxanone trimethylene carbonate, polyhybroxyalkonates (e.g., poly(hydroxybutyrate), poly (ethyl glutamate), poly (DTH iminocarbony (bisphenol A iminocarbonate), poly (ortho ester), and polycyanoacrylates.

In some aspects, the microcarriers comprise hydrogels, which are typically hydrophilic polymer networks filled with water. Hydrogels have the advantage of selective trigger of polymer swelling. Depending on the composition of the polymer network, swelling of the microparticle may be triggered by a variety of stimuli, including pH, ionic strength, thermal, electrical, ultrasound, and enzyme activities. Non-limiting examples of polymers useful in hydrogel compositions include, among others, those formed from polymers of poly(lactide-co-glycolide); poly(N-isopropylacrylamide); poly (methacrylic acid-g-polyethylene glycol); polyacrylic acid and poly(oxypropylene-co-oxyethylene) glycol; and natural compounds such as chrondroitan sulfate, chitosan, gelatin, fibrinogen, or mixtures of synthetic and natural polymers, for example chitosan-poly (ethylene oxide). The polymers may be crosslinked reversibly or irreversibly to form gels adaptable for forming three dimensional tissues.

In exemplary aspects, the microcarriers or beads for use in the present invention are composed wholly or composed partly of dextran.

In addition to the treatment of the tissue-culture surface, cells can require to be grown on coated surfaces, to enhance or improve their adhesion and/or spreading (i.e., using a coating). “Coating” as an additional surface treatment stands for all additional modifications made to increase cell adhesion in addition to the standard plasma or corona treatment which is performed on all cell culture plastic by manufacturer. Usually, coating is done with proteins or peptides. Various proteins can be used to coat tissue-culture treated dishes, including poly-L-Lysine, poly-D-Lysine, poly-Ornithine, gelatin, collagen I, IV, fibronectin, laminin, vitronectin, osteopontin, fibronectin domains, Matrigel™ (several components of the extracellular matrix with bound growth factors etc.), collagen gels, alginate gels, and lactate gels.

In one aspect, the two-dimensional culture system or microcarriers are coated. In various aspect, the PSCs are cultured on a laminin-coated surface.

In another aspect, expanding the cells includes culturing the cells on a non-coated surface. That is, after the initial differentiation step of the cells (i.e., while the PSCs are contacted with a GSK3β inhibitor), the cells are expanded in the absence of the GSK3β inhibitor, and on a surface that is not coated.

In the methods described herein, induced pluripotent stem cells are differentiated into MSCs. As used herein, “MSCs” refers to multipotent cells that express a combination of specific markers, that do not express markers that are specific to other differentiated or undifferentiated cells, and that can be further terminally differentiated into osteogenic, adipogenic or chondrogenic cells. For example, MScs are usually characterized as CD73, CD44 and CD90 positive adherent cells. MSCs are usually characterized as CD73, CD44 and CD90 positive and CD14, CD31, TRA 1-60, CD34, HLA-DR, SOX2, miR-302 HT, PECAM1, CDH5, CD45, MYH6 and SOX10 negative adherent cells. After about 6 days of culture under the conditions described herein, the MSCs are CD73, CD44 and CD90 positive and CD14, CD31, TRA 1-60, CD34, HLA-DR, SOX2, miR-302 HT, PECAM1, CDH5, CD45, MYH6 and SOX10 negative adherent cells.

In one aspect, the method described herein produces an enriched culture of MSCs. In some aspects, the enriched culture includes at least about 80% MSCs. For example, the enriched culture includes at least about 80%, 85%, 90%, 91%, 92%, 93%, 94%, 95%, 96%, 97%, 98%, 99% or more CD73, CD44 and CD90 positive MSCs. In one aspect, the enriched culture includes at least about 95% CD73, CD44 and CD90 positive and CD14, CD31, TRA 1-60, CD34, HLA-DR, SOX2, miR-302 HT, PECAM1, CDH5, CD45, MYH6 and SOX10 negative MSCs.

In one aspect, the MSCs generated are multipotent stem cells with osteogenic, adipogenic and chondrogenic differentiation potential.

By “differentiation potential”, it is meant that the MSCs obtained by the methods described herein can be terminally differentiated (using differentiation protocol well known in the art) into osteogenic, adipogenic and chondrogenic cells.

In another aspect, one differentiated into MSCs, the cells are expanded in a MSCs supporting culture media. There are several culture media that are known in the art as appropriate to support MSCs culture and expansion in vitro. The MSCs obtained with the methods described herein can be expanded in any MSCs supporting culture media. In one aspect, contacting and expanding the cells include contacting and expanding in serum-free culture conditions.

In another embodiment, the invention provides a method of generating mesenchymal stromal/stem cells (MSCs) including: (a) contacting an adherent culture of iPSCs with about 4 μM of a single GSK3β inhibitor for about 6 days; and (b) expanding the cells in culture from (a) in the absence of the GSK3β inhibitor thereafter, wherein after (b), a population of at least about 90% pure CD73+, CD44+ and CD90+MSCs is obtained, thereby obtaining MSCs.

In one embodiment, the invention provides a method of generating terminally differentiated osteogenic, adipogenic and/or chondrogenic from iPSCs including: a) generating MSCs cells by: (i) contacting an adherent culture of iPSCs with about 4 μM of a single GSK3β inhibitor for about 6 days; and (ii) expanding the cells in culture from (a) in the absence of the GSK3β inhibitor thereafter, wherein after (b), a population of at least about 90% pure CD73+, CD44+ and CD90+MSCs is obtained; and b) contacting the MSCs cells of a) with a mixture of agents to induce differentiation of the MSCs into terminally differentiated osteogenic, adipogenic and/or chondrogenic cells, thereby generating terminally differentiated osteogenic, adipogenic and/or chondrogenic cells.

Presented below are examples discussing methods of generating MSCs from iPSCs contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

Design of the Protocol to Differentiate iPSCs into MSCs

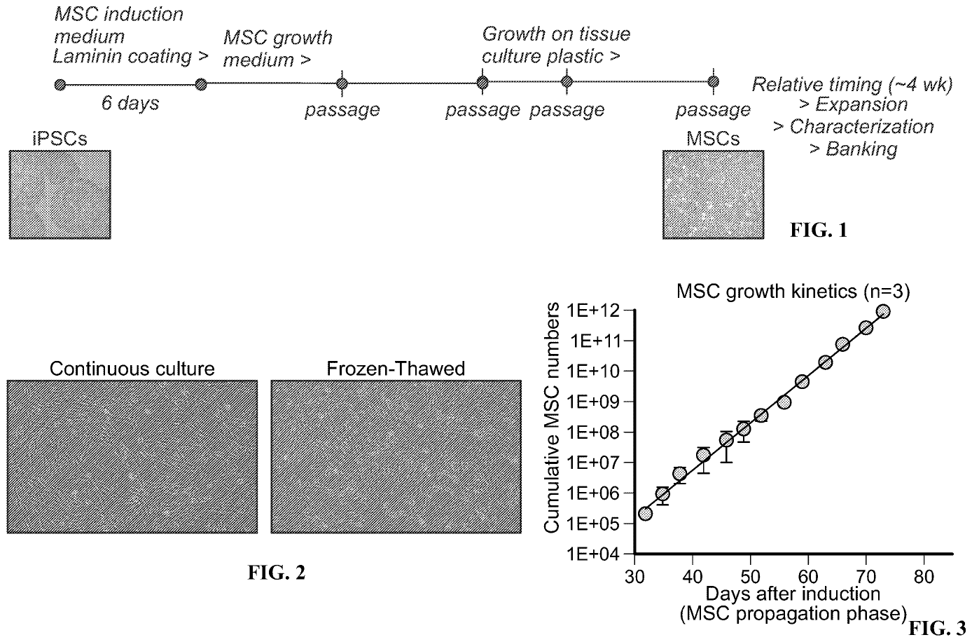

In a stepwise iterative developmental process, a new protocol promoting MSCs differentiation at high efficiency has been identified using a simple and GMP-compatible workflow (see FIG. 1).

The protocol involves the addition of a single factor, a GSK3b inhibitor, CHIR99021 during the initial days under adherent conditions either with or without serum. The hiPSCs were seeded on Laminin (iMatrix 511) in iPS-brew medium with Rock inhibitor. The next day, the medium was replaced with MSC induction medium (MSCI medium: iPS Brew medium with a GSK3b inhibitor, CHIR99021 (4 μM)). This treatment was continued for 6 days, with MSCI medium replacement every day. On the seventh day, the medium was replaced with MSC growth medium (MSCG), which can be serum-free (e.g., Milteny MSC medium) or with serum components (hPL or FCS)—an MSC-supportive medium in general. The hiPSC-MSCs generated by the method described herein contained the more than >90% of the cells expressing the MSC markers, CD73, CD90, CD105 and CD44 and less than 1-2% of the cells expressing endothelial cell markers (CD31), hematopoietic stem/progenitor markers (CD45 and CD34), immunogenic marker HLA-G, or the pluripotency marker TRA-1-60.

Following that protocol, MSCs were kept in culture for extended periods of time and maintained their phenotypic characteristics whether they were continuously cultured, or after being frozen and thawed (see FIGS. 2 and 3).

### Example 2

### Material and Methods

TABLE 1
Material for cell culture

| Material | Reference number/brief instructions |
|---|---|
| iMatrix-511 | Amsbio 892 011 |
| | Store at 2-8 °C Briefly centrifuge vial prior to use. |
| StemMACS iPS-Brew iMatrix- | Miltenyi Biotec 130-104-368 |
| 511XF | Thaw supplement at 37 °C, mix well, add to bottle of basal |
| | medium, mix well, aliquot at 45 ml, store at below −17 °C. |
| | For use, thaw 45 ml aliquot at 37 °C mix well by pipetting |
| | (no inversion). Optionally add 1:100 PS. Keep unused |
| | remainder at 2-8 °C up to 2 weeks. |
| Y-27632 (or Y, Rock inhibitor) | R&D TOCRIS 1254/50 |
| | Equilibrate vial to room temperature (RT) and briefly spin |
| | contents. Dissolve entire contents in DMSO to 10 mM (=1000×). |
| | Freeze aliquots in amber 1.5 ml tubes below −17 °C. |
| | For use, thaw aliquot at 37 °C. or RT and mix by |
| | flicking. Aliquots may be refrozen or stored at 2-8 °C |
| Accutase | Sigma A6964-500 ml |
| | Thaw at 37 °C, mix well, and prepare aliquots of 10-12 ml |
| | to be stored below −17 °C. For use, thaw aliquot at 37 °C and |
| | mix well. Accutase may be refrozen up to three times. |
| CHIR99021 (or CHIR) | STEMCELL Technologies 72054-10 mg |
| | Equilibrate vial to RT and briefly spin contents. Dissolve |
| | entire contents in DMSO to 3 mM. Freeze aliquots in amber |
| | 1.5 ml tubes below −17 °C. For use, thaw aliquot at 37 °C or |
| | RT and mix by flicking. Aliquots may be refrozen. |
| Penicillin/Streptomycin (Pen/Str, | ThermoFischer Scientific 15140122 - 100 ml |
| or PS) (100×) | Thaw, aliquot at 0.5 or 10 ml, and refreeze at < −17 °C |
| StemMacs MSC Expansion Media | Miltenyi Biotec 130-104-182 |
| Kit | Store at 2-8 °C |
| StemMacs MSC Expansion Media | Miltenyi Biotec 130-101-376 |
| Supplement | Store at −20 °C |
| | Thaw supplement at 37 °C, mix well, add to bottle of |
| | basal medium, mix well, aliquot at 45 ml, store at |
| | below −17 °C For use, thaw 45 ml aliquot at 37 °C |
| | mix well by pipetting (no inversion). Optionally add |
| | 1:100 PS. Keep unused remainder at 2-8 °C up to 1 |
| | week. |
| TrypLE | ThermoFisher A1285901 |
| | Store at 2-8 °C |
| StemPro Adipogenesis supplement | Gibco A10065-01 |
| | Store at −20 °C |
| StemPro Adipocyte differentiation | Gibco A10410-01 |
| basal media | Store at 2-8 °C |
| StemPro Osteogenesis supplement | Gibco A10066-01 |
| | Store at −20 °C |
| StemPro ® Osteocyte/Chondrocyte | Gibco A10069-01 |
| Differentiation Basal Medium | Store at 2-8 °C |
| StemPro ® Chondrogenesis | Gibco A10064-01 |
| Supplement | Store at −20 °C |
| Formaldehyde Solution | Sigma-Aldrich 47608 |
| | Store at 2-8 °C |
| Alzarin Red Solution | Sigma Aldrich 2003999 |
| | Store at 2-8 °C |
| Alcian-Blue Staining Solution | Merck TMS-010C |
| | Store at 2-8 °C |
| Oil Red O solution | Merck O1391 |
| | Store at room temperature |
| DMSO (1×) for cell culture | AppliChem A3672, 0250- 250 ml |
| | Store ready to use medium at RT. |
| EDTA Solution 0.5M | Biological Industries 01-862-1B |
| MSC Induction Medium (MSCI | For preparing 50 ml MI medium, adequate volume of |
| medium) | 3 mM CHIR99021 was added to a final concentration |
| | of 4 uM to CHIR99021 and 500 μl PS to StemMACS |
| | iPS-Brew XF. The Mesoderm Induction Medium was |
| | used at 37 °C to differentiate hiPSCs for the first 6 |
| | days. The StemMACS iPS-Brew XF with PS added |
| | (XF + PS), may be stored at 2-8 °C up to 2 weeks. |
| | However, CHIR99021 was added freshly every day, |
| | before replacing medium for the cells. |
| MSC growth Medium (MSCG | Miltenyi Biotec 130-104-182 & 130-101-376 |
| medium) | For preparing MSCG medium (complete medium), to |
| | 500 mL of the StemMACS MSC Expansion Medium |
| | XF Basal Media, add the thawed 7 mL StemMACS ™ |
| | MSC Expansion Media XF Supplement and 5 ml of |
| | PS. Use the complete MSCG medium within 1 week, |
| | when stored at 2-8 °C For longer storage, prepare |
| | aliquots and store at −20 °C |
| | Use the MSCG at 37 °C to differentiate the hiPSC- |
| | derived MSC progenitors from day 6 onwards. The |
| | MSCG medium is also used to further passage and |
| | maintain the hiPSC-derived MSCs. Store the MSCG |
| | medium at 2-8 °C up to 1 week. |
| Freezing medium | To prepare 10 ml of Freezing medium, add 1 ml |
| | DMSO to (10% DMSO) 9 ml MSCG and bring down |
| | its temperature to 2-8 °C in advance. |

TABLE 2
Antibodies used for Flow Cytometry (all to Store at 2-8 °C or, alternatively,
at −20 °C after addition of 80% (v/v) glycerol to 10% final):

| Antibody | Manufacturer |
|---|---|
| CD105 antibody | BioLegend 323202 |
| CD90 (Thy1) antibody | BioLegend 328135 |
| CD73 antibody | BD Biosciences BUV661 |
| CD14 MEM-15 antibody | Thermo fisher MA119223 |
| CD44 G-44-26 | BD Bioscience 555476 |
| CD31 (PECAM-1) antibody | BioLegend 303104 (FITC-labelled) |
| CD34 antibody | BD Bioscience 555821 (FITC-labelled) |
| IgG antibody | Life Technologies A11001 (Alexa Fluor 488-labelled) |
| IgG antibody | Life Technologies A21239 (Alexa Fluor 647-labelled) |
| CD45 antibody | Miltenyi Biotec 130-113-118 (FITC-labelled) |
| HLA-DR antibody | BD Bioscience 550853 (FITC-labelled) |
| TRA-1-60 antibody, REAfinity | Miltenyi Biotec 130-122-965 (PE, labelled) |
| IgG REAfinity | Miltenyi Biotec 130-113-438 (PE-labelled) |

### Methods

Thawing and Maintenance of hiPSCs:

The hiPSCs were thawed on Thursdays and passaged on Monday mornings and Thursday afternoons at a density of 200,000-250,000 per well of a 6-well plate. For the experiments, the hiPSCs were seeded on Thursdays and differentiations initiated on Fridays.

Thawing of hiPSCs:
Add PS to the iPS Brew XF medium (XF Medium) medium at 1:100 ratio (XF medium+PS). Add Rock inhibitor (Y-27632 (Y)) at 1:1000 concentration to XF medium+PS and mix.
Coat each well of a 6-well plate with 3 pl iMatrix-511 in 2 ml of the XF medium+PS+Y per well for at least 1 hour at 37 °C. Pre-warm the required volume of iPS Brew XF medium (XF Medium).
Add 5 ml XF medium+PS+Y medium to a 15 ml tube.
Transfer frozen cryovial to working area, ideally using a liquid nitrogen dewar, alternatively on dry ice. If walking distance is low (<~2 min) and within the same laboratory area
Thaw frozen cryovial of a given cell line in closed hand or 37 °C water bath. Once thawed, wipe tube with disinfectant wipe and transfer to the cell culture hood. Thawing process duration in maximum 4 min.
Open the cryotube and using a 1 ml pipette, gently pipet up and down once, then transfer the cell suspension to the 15 ml tube containing 5 ml of the XF medium+PS+Y medium. Close the 15 ml tube and centrifuge at 300×g for 1 min. In the meantime, label the lid of the new plate with cell line ID, passage number, date, operator initials, and other desired information.
Completely pipet/aspirate-off the supernatant. Using a 1 ml pipette, soak up ~300 μl of the XF medium+PS+Y medium from freshly prepared 6-well(s) and gently re-suspend cell pellet by pipetting up and down twice. Transfer cells to the new well(s) containing the XF medium+PS+Y medium drop-wise, place plate in the incubator, then distribute cell aggregates evenly in culture well(s) by agitating the plate in an infinity symbol-like manner (“∞”) for several rounds.
Next day (Friday), replace the medium with 4 ml of the XF medium+PS medium per well.
   Maintenance of hiPSCs:

### Monday

Pre-warm Accutase and the required volume of pre-warmed iPS Brew XF medium (XF Medium). Add PS to the XF medium at 1:100 ratio (XF medium+PS). Add Rock inhibitor (Y-27632 (Y)) at 1:1000 concentration to both Accutase and XF medium+PS and mix.
Coat the wells of a 6-well plate with 3 pl iMatrix-511 in 2 ml of the XF medium+PS+Y per well for at least 1 hour at 37 °C.
Treat the hiPSCs with 1 ml of Accutase+Y per well.
The hiPSCs should be 70-100% confluent and undifferentiated, before passaging or seeding for the experiment. Aspirate and completely remove the medium and wash the cells with 2 ml PBS per well. After removing PBS, add 1 ml of pre-warmed Accutase+Y and place the plate in the CO2 incubator for 10 min. Most cells should come-off by gently agitating the plate. If this is not the case, prolong digestion for 2 more minutes and so forth, until the cells lift-off virtually by themselves.
Add 1 ml (equal to the volume of Accutase+Y added) of prewarmed XF medium+PS+Y in a 15 ml tube. Transfer cells from all wells to this 15 ml tube. Centrifuge at 300 g for 3 minutes. Supernatant should be clear, and cells should form a compact pellet.
Remove the supernatant and re-suspend in 2 ml of pre-warmed XF medium+PS+Y per harvested well of a 6-well plate by pipetting 3 times up and down with a 1 ml pipette. Immediately collect 10 μl of the suspension to a counting chamber or cell counter and quantify the cell titer.
Seed 200,000 cells per well into each pre-coated well of a 6-well plate. Transfer the plate to the CO2 incubator and move the plate slowly in an infinity symbol pattern inside the incubator.

### Tuesday

Feed cells by replacing the medium with 2.5 ml pre-warmed prewarmed XF medium+PS per well.

### Wednesday

Feed cells by replacing the medium with 3 ml pre-warmed prewarmed XF medium+PS per well.

### Thursday

Confirm that the cells are sub-confluent and fully undifferentiated. In the afternoon, coat the wells with iMatrix-511 and passage the cells as above, at a density of 30,000 cells per well of a 6-well plate.
   Differentiation of hiPSCs into MSCs

### Friday (Day 0)

Confirm even distribution of hiPSCs of the wells to be used for differentiation. Cells should be flat and form loose colonies.
Prepare and/or pre-warm the MSCI medium medium. Thaw required aliquot of CHIR99021 (CHIR) at RT (2-5) min, then mix by flicking the tubes.
Rinse the cells once with 1 ml PBS at room temperature.
Aspirate the PBS and add 4 ml of the MSCI medium. Place the plate back into the CO2 incubator over the weekend.

### Monday (Day 3)

Replace the medium with freshly prepared pre-warmed MSCI medium at a ratio of 3 ml per well.

### Tuesday (Day 4)

Replace the medium with freshly prepared pre-warmed MSCI medium at a ratio of 3 ml per well.

### Wednesday (Day 5)

Replace the medium with freshly prepared pre-warmed MSCI medium at a ratio of 3 ml per well.
Collect the medium from the wells in 15 ml tubes. Add 1 ml of the pre-warmed MSCI medium to each well, to avoid cells from starving. Centrifuge the tubes with the collected medium for 1 minute at 300×G, so that any floating MSC progenitors are collected at the bottom of the tubes. Carefully aspirate the supernatant off from the centrifuged tubes, to retain only the cell pellet. Then, add adequate volume of pre-warmed MSCI medium (Volume=2×No. of wells from which medium was collected) from the sides of the tubes. Following this, re-suspend the cells with a 5 ml pipette gently by pipetting twice up and down, without disturbing and trying to preserve the clumps. Further, 2 ml of the re-suspended cells is added very gently to each well.

### Thursday (Day 6)

Collect the medium from the wells in 15 ml tubes. Add 1 ml of the freshly pre-warmed MSCG medium to each well, to avoid cells from starving. Centrifuge the tubes with the collected medium for 1 minute at 300×G, so that any floating MSC progenitors are collected at the bottom of the tubes. Carefully aspirate the supernatant off from the centrifuged tubes, to retain only the cell pellet. Then, add adequate volume of pre-warmed MSCG medium (Volume=2×No. of wells from which medium was collected) from the sides of the tubes. Following this, re-suspend the cells with a 5 ml pipette gently by pipetting twice up and down, without disturbing and trying to preserve the clumps. Further, 2 ml of the re-suspended cells is added very gently to each well.

### Friday (Day 7)

Replace the medium with 4 ml of the pre-warmed MSCG medium.

### Monday (Day 10)

Replace the medium with pre-warmed MSCG medium (3 ml per well).

### Tuesday (Day 11)

Replace medium.

### Wednesday (Day 12)

Replace the medium with pre-warmed MSCG medium (3 ml per well). The well will be ~80% confluent between 12-20 days, depending on the cell line. Till then, continue this schedule of treating the cells with the MSCG medium. Once the cells are more than 90% confluent, passage them at 1:2 ratio on Laminin-coated 6-well plates (Laminin: 3 pl iMatrix-511 in 2 ml of MSCG medium) (P1).
From the next passage (P2) onwards, the hiPSC-induced (iMSCs) are seeded on plastic. Maintain a passaging ratio of 1:2 for 3 passages, before diluting them. From passage 4 onwards, the cells can be diluted at 200K cells per well of a 6-well plate with 2 ml MSCG medium. Medium is replaced the next day after seeding and then, every 2 days. Every Friday, the MSCs are fed with 3-4 ml MSCG medium.
For the passaging of iPSC-MSCs, aspirate and discard the medium. For 1 well of a 6-well plate, rinse the cells with 1 ml PBS (−/−). After discarding the PBS, add 1 ml of 0.5 mM EDTA (prepared in PBS (−/−)) and incubate the cells in the incubator for 4 minutes. Then aspirate, and discard EDTA. Add 1 ml of TrypLE per well (6-well plate) and place the plate in the CO2 incubator for 4 minutes. Followed by this, use a 1 ml pipette to bring the cells into solution by pipetting up and down. Add in minimum 3 ml MSCG medium in a 15 ml tube. When the cells are in solution, pipette them for two times up and down and transfer into the 15 ml tube. Centrifuge at 300×G for 3 minutes, discard the supernatant and re-suspend the cell pellet with MSC medium by pipetting up and down, 4 to 5 times. After cell-counting, seed the accurate volume of the cell suspension on to plates or flasks at 1:2 ratio (till the 3rd passage) or at 20K cells/cm² (from passage 4 onwards).
   Cryopreservation and Long-Term Storage of the iPSC-Derived MSCs (iPSC-MSCs)

The iPSC-MSCs are stored for long-term at −196 °C, in the freezing medium.

Freezing of iPSC-MSCs:
Prepare the freezing medium and cool it down 2-8 °C in advance.
Prepare proper labels for the cryovials.
Harvest and count the MSCs, as done during the standard passaging.
Take accurate number of cells to be frozen in a tube and centrifuge at 300×G for 3 minutes. If the volume of the cell suspension is higher, increase the duration of centrifugation accordingly. After centrifugation, pipette/aspirate-out and discard the supernatant.
Re-suspend the cell pellet in adequate volume of the pre-cooled freezing medium. Try to prepare a suspension of homogenous single cells by pipetting up and down 1-2 times and distribute the accurate volume of the cell suspension in the freezing medium into the pre-labeled cryovials. 500,000 MSCs can be re-suspended in 1 ml of the freezing medium.
Transfer cryovials into Mr. Frosty™ Freezing Container and store them in −80 °C freezer to slowly cool down the temperature, very close to −1 °C/minute. It is recommended to transfer the cryovials into the liquid nitrogen tank from 24 h hours till one week post freezing procedure.
   Thawing of iPSC-MSCs:
Prepare a tube with accurate volume of the pre-warmed MSCG medium in advance.
Remove the cryovial from liquid nitrogen and immediately place it at 37 °C in a water bath, with intermittent shaking, until ≈80% of the cells are thawed, which takes less than 2 minutes.
Quickly pipette out the cell suspension in the cryovial into the MSCG medium in the tube and centrifuge at 300 g for 3 min.
Aspirate and discard the supernatant and re-suspend the pellet in 1-2 ml of the MSCG medium and transfer into a plate or a flask.
Then incubate the flask at 37 °C in a CO2 incubator for further MSC culture and maintenance.

### Analytical Methods:

### Flow Cytometry

For performing flow cytometry, cells are rinsed with PBS (1 ml per well of a 6-well plate).
Add 1 ml of 0.5 mM EDTA place the plates in the CO2 incubator for 4 minutes.
Discard the supernatant and add 1 ml of TrypLE and place the plate again for 4 minutes in the CO2 incubator.
Add 2-3 ml medium in a 15 ml tube and transfer the cells into this tube.
Transfer to 15 ml tubes and centrifuge at 300×G for 2.0 to 3.0 minutes.
Aspirate and discard the medium and add 1 ml of PBS to each tube.
Vortex shortly and centrifuge at 300×G for 2.0 minutes.
Remove the supernatant and re-suspend the cells in adequate volume of PBS, so that 1 million cells are included in 100 μl PBS. Distribute 100 μl into each 1.5 ml tube. If the number of cells is less, a minimum of 200,000 cells should be included in 100 μl PBS for each antibody.
Add 1.0-5.0 μl of each antibody (depends on the concentration of the antibody) for staining, into the 100 μl cell suspension, vortex each tube shortly for 5 seconds and incubate for 20 min at room temperature in the dark.
After incubation, add 300 μl of PBS and centrifuge at 400×g for 1 min. Discard the supernatant.
If the primary antibodies are not pre-stained, add 100 μl of PBS to the pellets, add adequate volume of the secondary antibodies (final concentration, according to the manufacturer's instructions). Then, vortex the tubes shortly for 5 seconds each and incubate for 20 minutes. After incubation, add 300 μl of PBS and centrifuge at 400×g for 1 min. Discard the supernatant. Add 300 μl of PBS to each tube, resuspend by vortexing shortly for 5 seconds and perform flow cytometry.
If the primary antibodies are pre-stained, add 300 μl of PBS to each tube, resuspend by vortexing shortly for 5 seconds and perform flow cytometry.
After the detection is completed, save the file on the G-drive, and analyze the flow cytometry result with the analysis software.

### Differentiation of MSCs

### Osteogenic Differentiation:

The iPSC derived MSCs are seeded in a 12 well plate (plastic) at 10K cells/cm² in XF medium+PS and incubate overnight in a CO2 incubator.
The next day, replace the medium with the pre-warmed StemPro Osteogenesis medium (22.25 ml StemPro® Osteocyte/Chondrocyte Differentiation Basal Medium+2.5 ml StemPro Osteogenesis supplement+250 μl PS) and continue the incubation in CO2 incubator. MSCs will expand as they differentiate in the osteogenesis medium.
Replace with fresh pre-warmed Osteogenesis medium every 3-4 days. On day 21 or later, osteogenesis can be observed using the Alizarin Red S stain analysis.

### Alizarin Red S Stain Analysis

Remove the media from the wells of the 12-well plate.
Rinse once with PBS (1 ml).
Remove the PBS from the wells
Fix cells with 4% formaldehyde solution (diluted in PBS) for 30 minutes.
After fixation, rinse twice with distilled water and stain the cells with 2% Alizarin Red S solution for 2-3 minutes on a plate shaker at room temperature.
Rinse well 3-4 times with 2 ml deionized water, visualize under light microscope and capture the images.

### Adipogenic Differentiation:

The iPSC derived MSCs are seeded in a 12 well plate (plastic) at 80K cells/cm² in XF medium+PS and incubate overnight in a CO2 incubator.
The next day, replace the medium with the pre-warmed StemPro Adipogenesis medium (22.25 ml StemPro® Adipocyte Differentiation Basal Medium+2.5 ml StemPro Adipogenesis supplement+250 μl PS) and continue the incubation in CO2 incubator. MSCs will expand as they differentiate in the adipogenic conditions.
Replace with fresh pre-warmed Adipogenesis medium every 3-4 days. On day 21 or later, adipogenesis can be observed using the Oil Red 0 stain analysis.

### Oil Red O Stain Analysis

Remove the media from the wells of the 12-well plate.
Rinse once with PBS (1 ml).
Remove the PBS from the wells
Fix cells with 4% formaldehyde solution (diluted in PBS) for 30 minutes.
After fixation, rinse twice with distilled water and stain the cells with Oil Red 0 solution (6 ml Oil Red 0 stock solution+4 ml deionized water) for 20 minutes on a plate shaker at room temperature.
Rinse well 3-4 times with 2 ml deionized water, visualize under light microscope and capture the images.

### Chondrogenic Differentiation:

The iPSC derived MSCs are resuspended in XF medium+PS in a cell concentration of 1.6×107 viable cells/ml.
Generate micro mass cultures by seeding 5 μl droplets of cell solution in the center of multi-well plate wells (5 droplets in a 12-well)
After cultivation micro mass cultures in minimum for 2 hours at 37 °C incubator with 5% CO2, add warmed chondrogenesis media to culture vessels and incubate again at 37 °C incubator with 5% CO2
Refeed cultures every 2-3 days
At 14 day or later, chondrogenesis can be observed using the Alcian blue stain analysis.

### Alcian Blue Stain Analysis

After the differentiation time is completed, carefully remove the medium.
Gently wash the spheroids with PBS (1 ml).
Remove the PBS from the tubes.
Fix cells with 4% formaldehyde solution (diluted in PBS) for 3 hours.
After fixation, transfer the spheroids to well-labelled plates. This makes it easier to handle the spheroids.
Aspirate the fixative and wash twice with distilled water
stain the cells with Alcian blue staining solution and incubate in dark for 45 minutes.
Carefully remove the Alcian blue staining solution with a 1 ml pipette, retaining the spheroid.
Wash twice with deionized water for 10 minutes each.
Add PBS to the spheroids, analyze the cartilage spheroids and capture images. The cartilage spheroids will stain dark blue, and the negative control will stain light blue.

### Example 3

Generation and Characterization of Natural Killer Cells Differentiated from Human Pluripotent Stem Cells

The invention is broadly relevant to the field of iPSC-based applications such as cell therapy, targeting MSCs as a differentiation product. The procedure facilitates their derivation and should, particularly in cell therapy, enable more robust manufacturing processes under clean room conditions as it avoids complex handling procedures and because it is based on a simple and defined iPSC treatment protocol. The novel approach that was developed for differentiating MSCs cells from iPSCs that allows for higher yields, higher purity and increased simplicity compared to existing methods. This was achieved by replacing the laborious and undefined EB phase with the directed differentiation of iPSCs into MSCs that further differentiate into osteogenic, adipogenic and chondrogenic cells. The protocol has been developed with a GMP-compliant process in mind and will allow for off-the-shelf MSCs derived from HLA-homozygous iPSC banks.

iPSCs were maintained and expanded as described in Examples 1-2 and differentiated in to MSCs following the methods described in Example 2 and as illustrated in FIG. 1.

From day 6 onwards, a culture media suitable for MSCs is used without GSK3β inhibitor. As illustrated in FIGS. 2 and 3, MSCs obtained by the method described herein displayed continuous exponential growth and retained they phenotype even after being frozen and thawed.

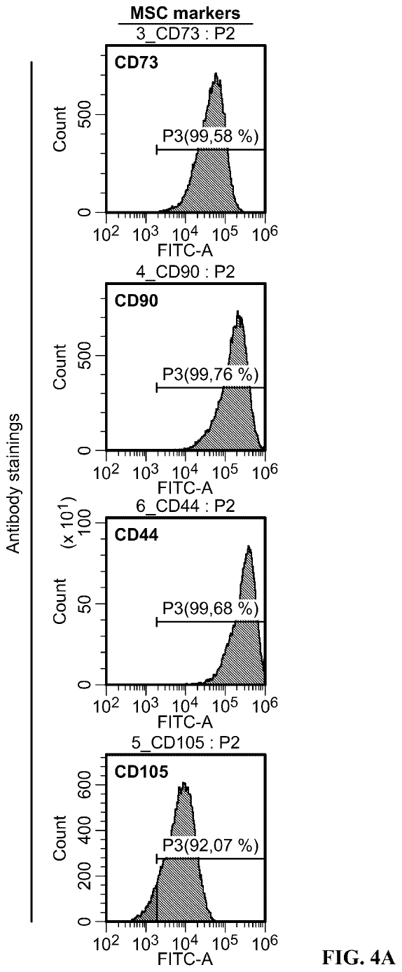

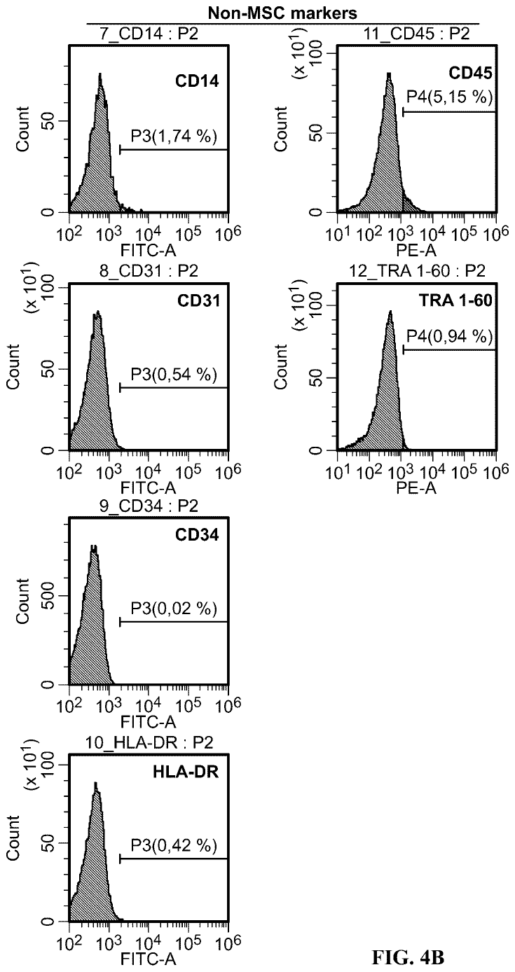

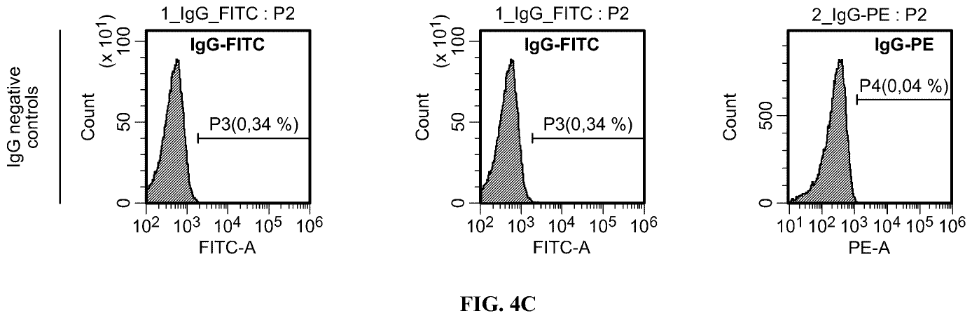

The characterization of the MSCs included analysis of MSCs specific biomarkers by flow cytometry. As shown in FIG. 4A, the MSCs obtained displayed CD73, CD90 and CD44 expression at more than 99%, and CD105 at about 82%. As further illustrated in FIG. 4B, it was demonstrated that the MSCs did not express non-MSC markers such as CD14, Cd45, CD31, CD34, TRA 1-60 or HLA-DR. The specificity of the analysis was confirmed by the negative control (FIG. 4C).

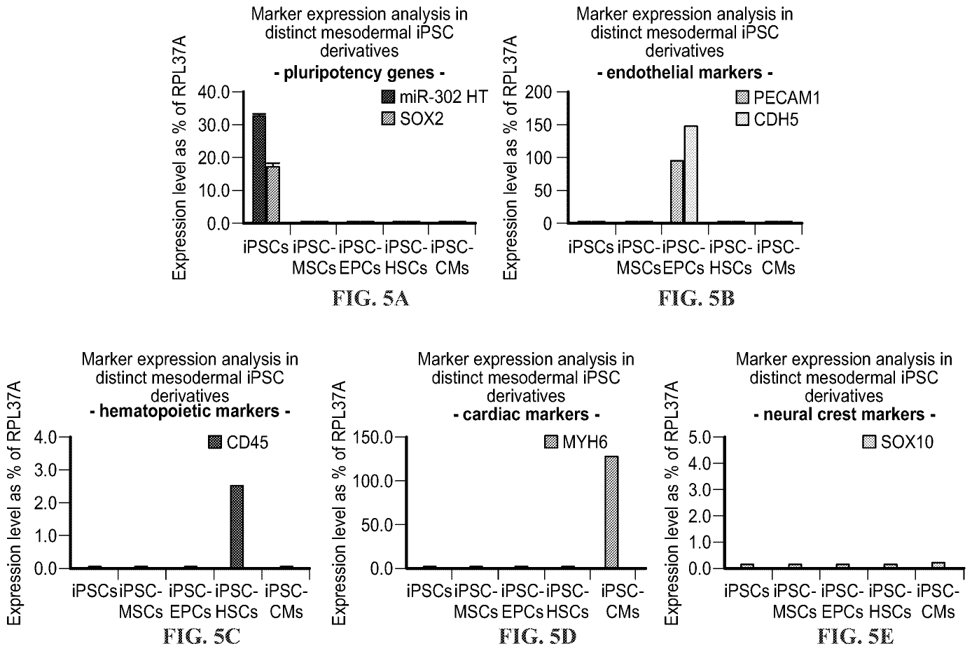

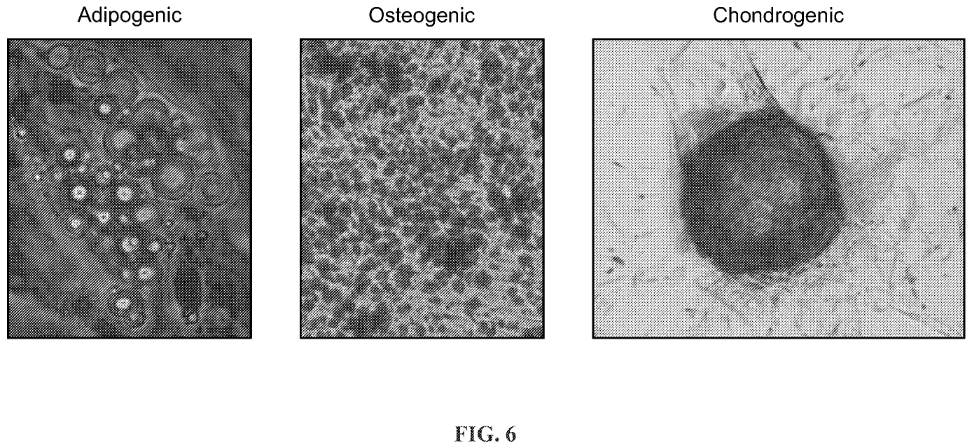

The characterization of the cells further included an analysis of the expression of pluripotency genes, such as miR-302 HT and SOX2; endothelial markers, such as PECAM and CDH5; hematopoietic marker, such as CD45; cardiac marker such as MYH6; and neural crest markers, such as SOX10, by RTqPCR. As shown in FIGS. 5A-5E, MSCs were negative for all of those non-MSCs markers.

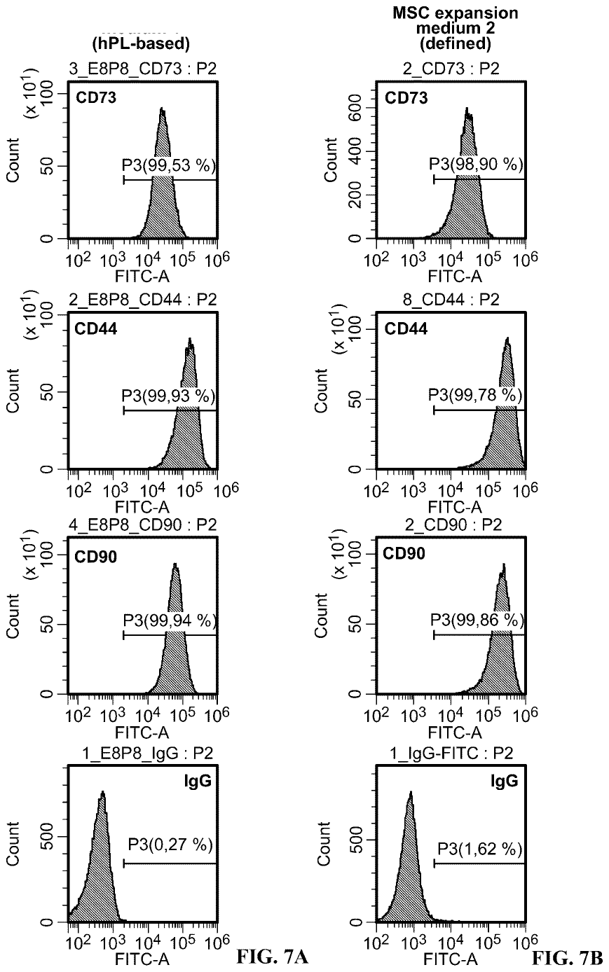

MSCs were further analyzed to assess whether the culture media in which the cells are expanded could affect their phenotype. As shown in FIGS. 7A-7B, the expression of CD73, CD44 and CD90 was assess in MSCs maintained and expanded in two different MSCs culture media. there was no effect of the culture media on the expression of the MSCs markers.

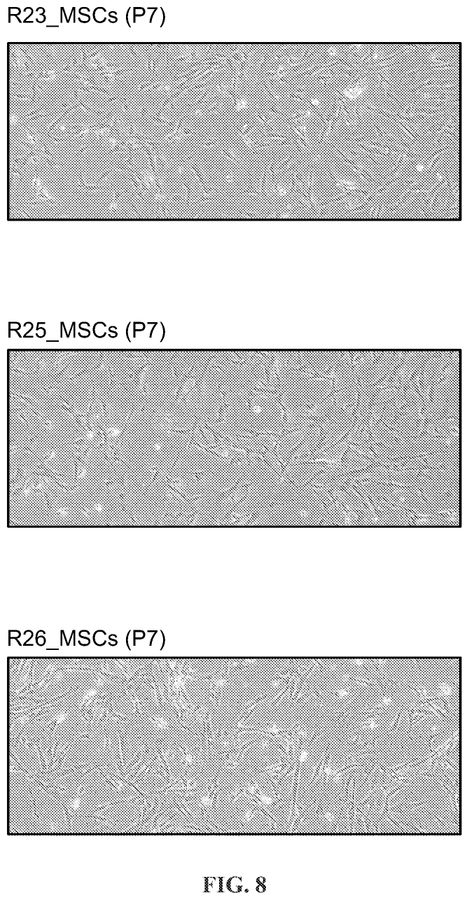

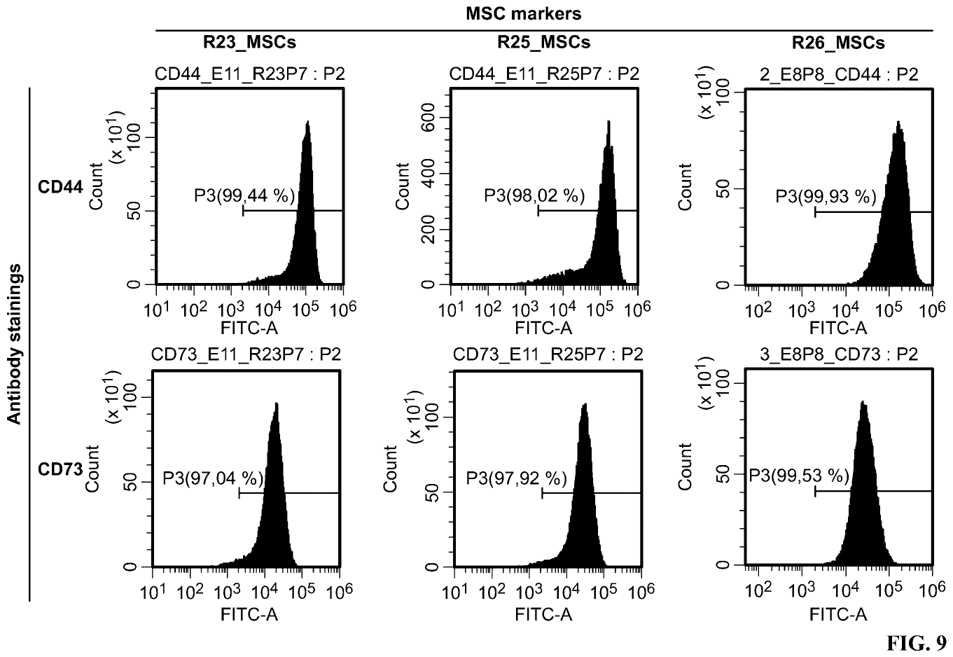

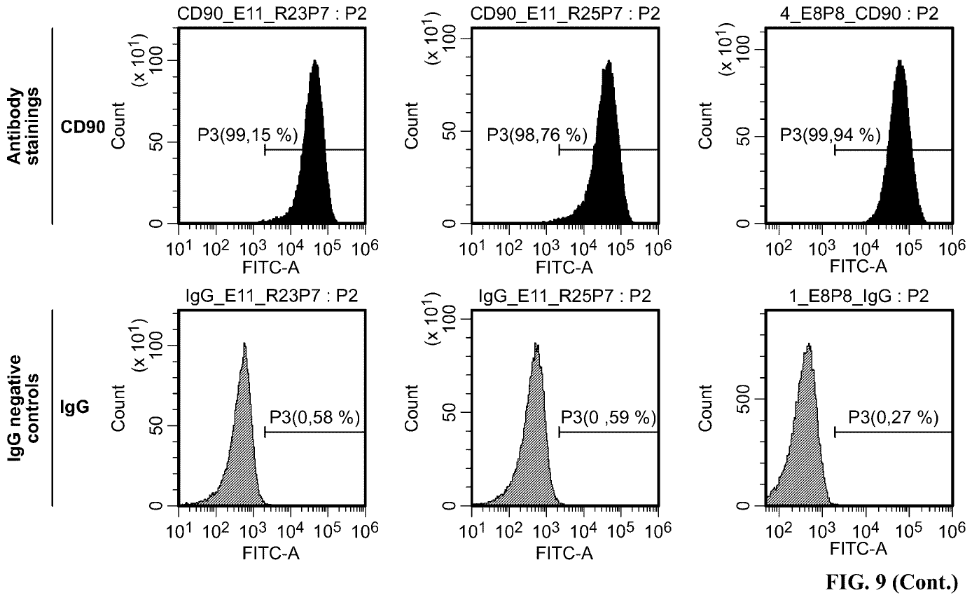

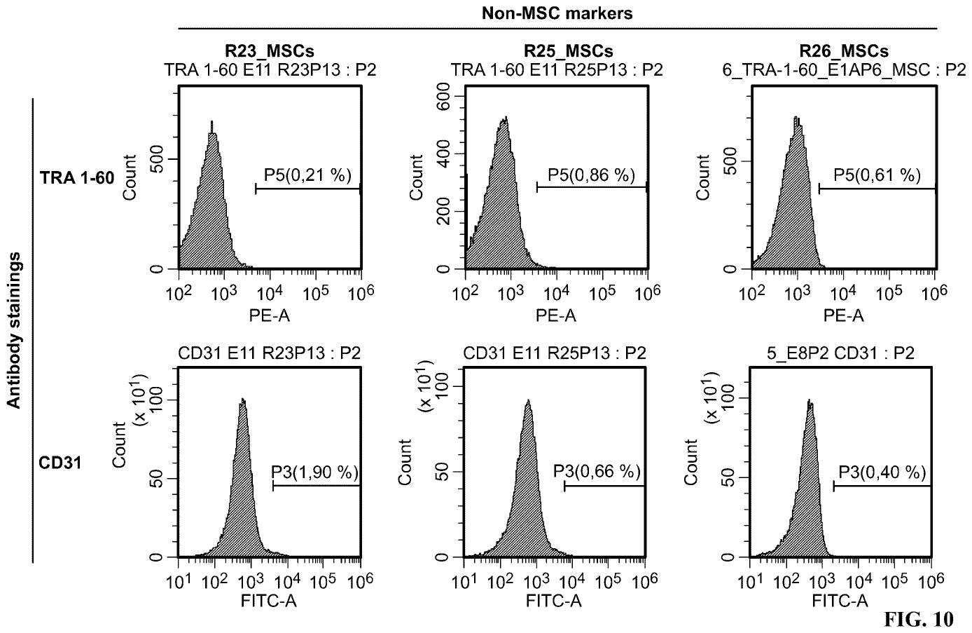

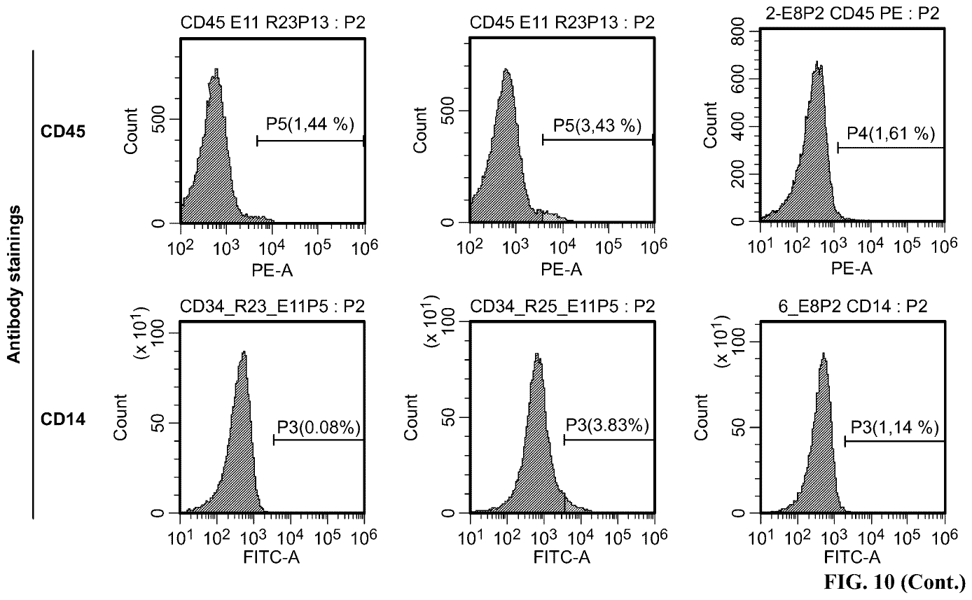

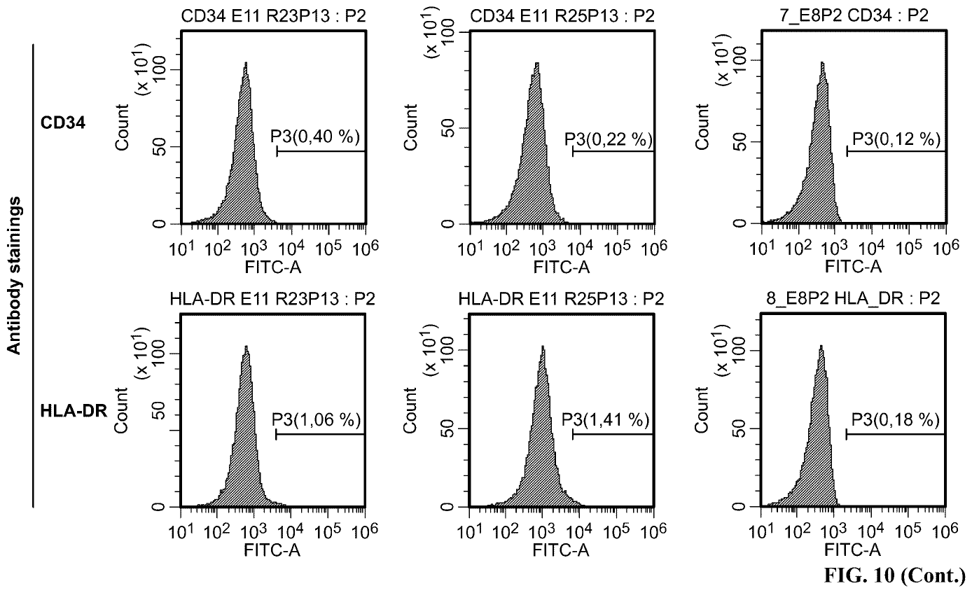

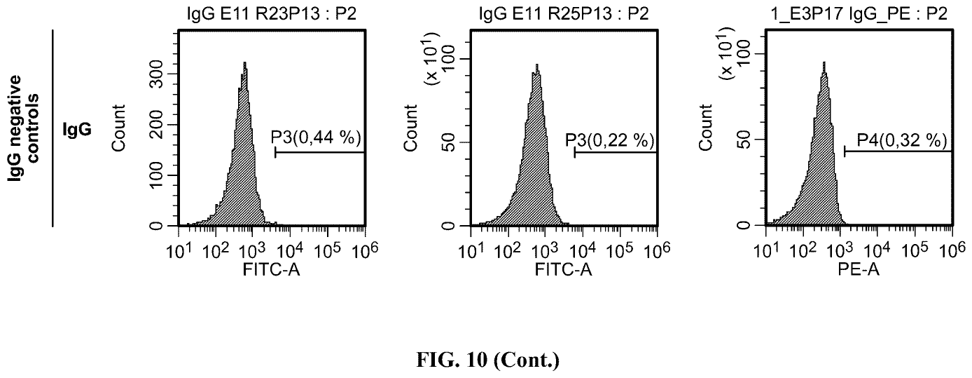

The reproducibility of the methods was assessed, and the phenotype and expression of MSCs markers were analyzed in MSCs derived from several independent iPSCs lines. As shows in FIGS. 8, 9 and 10, regardless of the iPSC line used, the MSCs obtained retained their phenotype (adherent to plastic in culture), and their strong expression of CD44, CD73 and CD90, while remaining negative for non-MSCs markers such as TRA 1-60, CD31. CD45, Cd14, Cd34 and HLA-DR.

### REFERENCES

Jiang Y, Jahagirdar B N, Reinhardt R L, Schwartz R E, Keene C D, Ortiz-Gonzalez X R, Reyes M, Lenvik T, Lund T, Blackstad M, Du J, Aldrich S, Lisberg A, Low W C, Largaespada DA, Verfaillie C M. Pluripotency of mesenchymal stem cells derived from adult marrow. Nature. 2002 Jul. 4; 418(6893):41-9.
Pittenger M F, Mackay A M, Beck S C, Jaiswal R K, Douglas R, Mosca J D, Moorman M A, Simonetti D W, Craig S, Marshak D R. Multilineage potential of adult human mesenchymal stem cells. Science. 1999 Apr. 2; 284(5411):143-7.
Lalu M M, Mazzarello S, Zlepnig J, Dong Y Y R, Montroy J, McIntyre L, Devereaux P J, Stewart D J, David Mazer C, Barron C C, McIsaac D I, Fergusson D A. Safety and Efficacy of Adult Stem Cell Therapy for Acute Myocardial Infarction and Ischemic Heart Failure (SafeCell Heart): A Systematic Review and Meta-Analysis. Stem Cells Transl Med. 2018 December; 7(12):857-866.
Eom Y W, Shim K Y, Baik S K. Mesenchymal stem cell therapy for liver fibrosis. Korean J Intern Med. 2015 September; 30(5):580-9.
Jin M C, Medress Z A, Azad T D, Doulames V M, Veeravagu A. Stem cell therapies for acute spinal cord injury in humans: a review. Neurosurg Focus. 2019 Mar. 1; 46(3): E10.
Cortez-Toledo E, Rose M, Agu E, Dahlenburg H, Yao W, Nolta J A, et al. Enhancing retention of human bone marrow mesenchymal stem cells with prosurvival factors promotes angiogenesis in a mouse model of limb ischemia. Stem Cells Dev. 2019; 28(2):114-9.
Dominici M, Le Blanc K, Mueller I, Slaper-Cortenbach I, Marini F, Krause D, Deans R, Keating A, Prockop Dj, Horwitz E. Minimal criteria for defining multipotent mesenchymal stromal cells. The International Society for Cellular Therapy position statement. Cytotherapy. 2006; 8(4):315-
Gomez-Salazar M, Gonzalez-Galofre Z N, Casamitjana J, Crisan M, James A W, Peault B. Five Decades Later, Are Mesenchymal Stem Cells Still Relevant?Front Bioeng Biotechnol. 2020 Feb. 28; 8:148.
Jin W, He Y, Li T, Long F, Qin X, Yuan Y, Gao G, Shakhawat H M, Liu X, Jin G, Zhou Z. Rapid and robust derivation of mesenchymal stem cells from human pluripotent stem cells via temporal induction of neuralized ectoderm. Cell Biosci. 2022 Mar. 15; 12(1):31.
Sánchez L, Gutierrez-Aranda I, Ligero G, Rubio R, Muñoz-López M, García-Pérez J L, Ramos V, Real P J, Bueno C, Rodriguez R, Delgado M, Menendez P. Enrichment of human ESC-derived multipotent mesenchymal stem cells with immunosuppressive and anti-inflammatory properties capable to protect against experimental inflammatory bowel disease. Stem Cells. 2011 February; 29(2):251-62. doi: 10.1002/stem.569.
Zhao Q, Gregory C A, Lee R H, Reger R L, Qin L, Hai B, Park M S, Yoon N, Clough B, McNeill E, Prockop D J, Liu F. MSCs derived from iPSCs with a modified protocol are tumor-tropic but have much less potential to promote tumors than bone marrow MSCs. Proc Natl Acad Sci USA. 2015 Jan. 13; 112(2):530-5.
Hawkins K E, Corcelli M, Dowding K, Ranzoni A M, Vlahova F, Hau K L, Hunjan A, Peebles D, Gressens P, Hagberg H, de Coppi P, Hristova M, Guillot P V. Embryonic Stem Cell-Derived Mesenchymal Stem Cells (MSCs) Have a Superior Neuroprotective Capacity Over Fetal MSCs in the Hypoxic-Ischemic Mouse Brain. Stem Cells Transl Med. 2018 May; 7(5):439-449. doi: 10.1002/sctm.17-0260.
Leyendecker Junior A. TGF-β Inhibitor SB431542 Promotes the Differentiation of Induced Pluripotent Stem Cells and Embryonic Stem Cells into Mesenchymal-Like Cells. Stem Cells Int. 2018 Jul. 2; 2018:7878201. doi: 10.1155/2018/7878201.
Karlsson C, Emanuelsson K, Wessberg F, Kajic K, Axell M Z, Eriksson P S, Lindahl A, Hyllner J, Strehl R. Human embryonic stem cell-derived mesenchymal progenitors—potential in regenerative medicine. Stem Cell Res. 2009 July; 3(1):39-50.
Sheyn D, Ben-David S, Shapiro G, De Mel S, Bez M, Ornelas L, Sahabian A, Sareen D, Da X, Pelled G, Tawackoli W, Liu Z, Gazit D, Gazit Z. Human Induced Pluripotent Stem Cells Differentiate into Functional Mesenchymal Stem Cells and Repair Bone Defects. Stem Cells Transl Med. 2016 November; 5(11):1447-1460.
Giuliani M, Oudrhiri N, Noman Z M, Vernochet A, Chouaib S, Azzarone B, Durrbach A, Bennaceur-Griscelli A. Human mesenchymal stem cells derived from induced pluripotent stem cells down-regulate NK-cell cytolytic machinery. Blood. 2011 Sep. 22; 118(12):3254-62.
Liu Y, Goldberg A J, Dennis J E, Gronowicz G A, Kuhn L T. One-step derivation of mesenchymal stem cell (MSC)-like cells from human pluripotent stem cells on a fibrillar collagen coating. PLoS One. 2012; 7(3): e33225.

Although the invention has been described with reference to the above examples, it will be understood that modifications and variations are encompassed within the spirit and scope of the invention. Accordingly, the invention is limited only by the following claims.

What is claimed is:

1. A method of generating mesenchymal stromal/stem cells (MSCs) comprising:
(a) contacting an adherent culture of pluripotent stem cells (PSCs) with a WNT signaling pathway activator for about 4-8 days; and
(b) expanding the cells in culture from (a) in the absence of the WNT signaling pathway activator thereafter,
thereby generating MSCs.

2. The method of claim 1, wherein the WNT signaling pathway activator is a GSK3β inhibitor.

3. The method of claim 2, wherein the GSK3β inhibitor is CHIR99021.

4. The method of claim 3, wherein the PSCs are contacted with about 2-6 μM of CHIR99021.

5. The method of claim 3, wherein the PSCs are contacted with about 4 μM of CHIR99021.

6. The method of claim 3, wherein the PSCs are contacted with CHIR99021 for about 6 days.

7. The method of claim 3, wherein the PSCs are contacted with about 4 μM of a CHIR99021 for about 6 days.

8. The method of claim 1, wherein contacting comprises incubating the PSCs with fewer than three WNT signaling pathway activators.

9. The method of claim 1, wherein contacting comprises incubating the PSCs with a single WNT signaling pathway activator.

10. The method of claim 1, wherein prior to (a), the iPSCs are maintained in a culture media comprising a ROCK inhibitor.

11. The method of claim 8, wherein the ROCK inhibitor is Y-27632.

12. The method of claim 1, wherein the PSCs are cultured on a laminin-coated surface.

13. The method of claim 1, wherein expanding the cells comprises culturing the cells on a non-coated surface.

14. The method of claim 1, wherein the method produces an enriched culture of MSCs.

15. The method of claim 12, wherein the enriched culture comprises at least about 90% MSCs.

16. The method of claim 12, wherein the enriched culture comprises at least about 95% MSCs.

17. The method of claim 1, wherein the MSCs are CD73, CD44 and CD90 positive.

18. The method of claim 1, wherein the MSCs are CD14, CD31, TRA 1-60, CD34, HLA-DR, SOX2, miR-302 HT, PECAM1, CDH5, CD45, MYH6 and SOX10 negative.

19. The method of claim 1, wherein the MSCs generated are multipotent stem cells with osteogenic, adipogenic and chondrogenic differentiation potential.

20. The method of claim 1, wherein expanding the cells comprises culturing the cells in a MSCs supporting culture media.

21. The method of claim 1, wherein contacting and expanding the cells comprise contacting and expanding in serum-free culture conditions.

22. The method of claim 1, wherein the culture of PSCs does not comprise embryoid bodies.

23. The method of claim 1, wherein the WNT signaling pathway activator is a WNT ligand, a recombinant protein, an inhibitor of a WNT signaling pathway inhibitor or an indirect WNT activator.

24. The method of claim 1, wherein the PSCs are human pluripotent stem cells (hPSCs).

25. The method of claim 24, wherein the hPSCs are induced pluripotent stem cells (hiPSCs) or human embryonic stems cells (hESCs).

26. The method of claim 1, wherein the PSCs are iPSCs.

27. A method of generating mesenchymal stromal/stem cells (MSCs) comprising:
(a) contacting an adherent culture of induced pluripotent stem cells (iPSCs) with about 4 μM of a single GSK3β inhibitor for about 6 days; and
(b) expanding the cells in culture from (a) in the absence of the GSK3β inhibitor thereafter,
wherein after (b), a population of at least about 90% pure CD73+, CD44+ and CD90+MSCs is obtained,
thereby obtaining MSCs.
